(19) United States
(12) Patent Application Publication
(10) Pub. No.: US 2022/0047296 A1
(43) Pub. Date: Feb. 17, 2022

(54) Obturator to Facilitate Catheter Line Draw

(71) Applicant: Becton, Dickinson and Company, Franklin Lakes, NJ (US)

(72) Inventors: S. Ray Isaacson, Layton, UT (US); Jonathan Karl Burkholz, Salt Lake City, UT (US); Megan Scherich, Salt Lake City, UT (US); Curtis H. Blanchard, Riverton, UT (US); John Lackey, West Valley City, UT (US); Benjamin Hopwood, Salt Lake City, UT (US)

(21) Appl. No.: 17/401,493

(22) Filed: Aug. 13, 2021

Related U.S. Application Data
(60) Provisional application No. 63/065,773, filed on Aug. 14, 2020.

(51) Int. Cl. A61B 17/34; A61B 17/00

(52) U.S. Cl. CPC A61B 17/3417; A61B 2017/00336; A61B 17/00234

(57) ABSTRACT

A vascular access device to passively open a fluid path within a vasculature. The vascular access device may include a catheter and an obturator slidably positioned within the lumen of the catheter. The catheter may have a proximal end, a distal tip comprising an opening having an inner diameter, a sidewall defining a lumen between the proximal end and distal tip. The obturator may have a distal end with an outer diameter configured to form an interference fit with the inner diameter of the distal tip. The obturator may be positioned within the lumen to open various fluid pathways. The obturator may be repositioned to move the catheter tip.

### CROSS-REFERENCE TO RELATED APPLICATION

The present application claims priority to U.S. Provisional Application Ser. No. 63/065,773, entitled “Obturator to Facilitate Catheter Line Draw”, filed Aug. 14, 2020, the entire disclosure of which is hereby incorporated by reference in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal end. The catheter and the introducer needle may be assembled so that the distal end of the introducer needle extends beyond the distal end of the catheter with the bevel of the needle facing up away from skin of the patient. The catheter and the introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Catheter functionality, however, may be impeded for several reasons, particularly when there is a prolonged dwelling time of the catheter within the vasculature. For example, when the catheter is left inserted in the patient for more than a day, the catheter may become susceptible to complications and obstructions that impede fluid flow. For example, the catheter may become occluded at a tip of the catheter due to the presence of fibrin sheath, thrombus, vein walls, or valves. Further, if the catheter is fenestrated (i.e., having holes near the tip to decrease fluid velocity at the tip and improve blood draw success), the catheter tends to become occluded faster than a non-fenestrated catheter.

The subject matter claimed herein is not limited to embodiments that solve any disadvantages or that operate only in environments such as those described above. Rather, this background is provided to illustrate one example technology area where some implementations described herein may be practiced.

### SUMMARY

The present disclosure relates generally to vascular access devices, systems, and methods to infuse fluids and/or collect blood from the vasculature of a patient. In particular, the present disclosure relates to systems, devices and methods to facilitate catheter functionality and blood draw success. In some embodiments, various features of a vascular access device may facilitate one or more of the following: repositioning a distal tip of a catheter within the vasculature to open a fluid path, selectively opening and closing the distal tip, and selectively opening and closing one or more fenestrations.

In some embodiments, the vascular access device may include the catheter and an obturator slidably positioned within the lumen of the catheter. In some embodiments, the catheter may include a proximal end, a distal tip, a sidewall defining the lumen between the proximal end and the distal tip, and a first fluid pathway through the opening. In some embodiments, the distal tip may include an opening. In some embodiments, the sidewall of the catheter may include a fenestration forming a second fluid pathway, which may extend through the fenestration.

In some embodiments, the obturator may include a distal end with an outer diameter configured to form an interference fit with an inner diameter of the distal tip of the catheter, which may correspond to an inner diameter of the opening of the distal tip. In some embodiments, the obturator may include a tubular shaft and a third fluid pathway extending through a length of the tubular shaft.

In some embodiments, the obturator may be positioned with the lumen of the catheter such that a fluid is blocked or prevented from flowing through the second fluid pathway. In some embodiments, the obturator may be positioned within the lumen of the catheter such that a fluid is permitted to flow through the second fluid pathway. In some embodiments, a fluid is permitted to flow through the first and third fluid pathways. In some embodiments, a particular fluid pathway, such as, for example, the first fluid pathway or the second fluid pathway, can be cleared by repositioning the obturator within the lumen of the catheter.

In some embodiments, an obturator may include the tubular shaft having a hole formed through a sidewall of the tubular shaft and a fourth fluid pathway through the hole. In some embodiments, the obturator may be positioned within the lumen of the catheter such that a fluid is permitted to flow through the second and fourth fluid pathways. In some embodiments, the obturator may be positioned within the lumen of the catheter such that a fluid is blocked or prevented from flowing through the second and fourth fluid pathways. In some embodiments, a fluid is permitted to flow through the first and third fluid pathways. In some embodiments, moving or repositioning the obturator within the lumen of the catheter causes a distal tip of the catheter to move.

In some embodiments, the distal end of the obturator may be solid or closed such that a fluid is blocked or prevented from flowing through the distal end of the obturator. In some embodiments, the obturator may be positioned within the lumen of the catheter such that a fluid is blocked or prevented from flowing through the first or second fluid pathways. In some embodiments, the obturator may be positioned within the lumen of the catheter such that a fluid flows through the second fluid pathway. In some embodiments, the obturator may be positioned within the lumen of the catheter such that a fluid is permitted to flow through the first and second fluid pathways.

In some embodiments, an obturator may include a body with an outer diameter configured to slide within the lumen of a catheter. In some embodiments, the outer diameter of the body forms the interference fit with the inner diameter of the distal tip of a catheter. In some embodiments, the body is significantly smaller than the inner diameter of the catheter of the distal tip of the catheter. In some embodiments, the body is rounded or spherical to prevent injury to vasculature when the body is moved distal the distal tip of the catheter. In some embodiments, the body is a soft spring with good compliance. In some embodiments, the obturator further may include a guidewire attached to a distal end of the body. In some embodiments, the guidewire attaches to a place other than the center of the body.

In some embodiments, the guidewire may include a shaped portion with a maximum height that is greater than the inner diameter of the distal tip of the catheter. In some embodiments, the shaped portion of the guidewire is configured to contact an inner wall surface forming the lumen of the catheter to temporarily deform a static conformation of the catheter at a point of contact between the shaped portion of the guidewire and the inner wall surface forming the lumen of the catheter.

In some instances, the obturator may cause the distal tip of the catheter to move away from an inner sidewall of a patient's vein. In some instances, moving or repositioning the obturator causes the distal tip of the catheter to move away from an object or surface occluding the distal tip of the catheter. In some embodiments, the obturator may be positioned within the lumen of the catheter such that a fluid is blocked or prevented from flowing through the first fluid pathway. In some embodiments, the obturator may be positioned within the lumen of the catheter such that a fluid is permitted to flow through the first fluid pathway.

In some embodiments, moving or repositioning the guidewire distally, proximally, and/or rotationally causes the distal tip of the catheter to move. In some embodiments, the body of the obturator may include a tubular shaft and a third fluid pathway extending through a length of the tubular shaft. In some embodiments, the body of the obturator comprises a hole in a sidewall of the tubular shaft and a fourth fluid pathway through the hole. In some embodiments, the body comprises a proximal portion with an outer diameter configured to form an interference fit with the inner diameter of a distal tip of a catheter and a distal portion with an outer diameter that is larger than the outer diameter of the proximal portion. In some embodiments, the body comprises a metal part and a plastic part. In some embodiments, the guidewire attaches to the metal part. In some embodiments, the guidewire is welded to the metal part.

In some embodiments, moving or repositioning the guidewire of the obturator moves or repositions the body of the obturator. In some embodiments, moving or repositioning the guidewire of the obturator moves or repositions the body of the obturator, which in turn moves or repositions the distal tip of the catheter in which the obturator may be positioned. In some embodiments, pulling the guidewire proximally causes bending of the catheter, thereby moving the tip away from occlusions. In some embodiments, rotating the guidewire rotates the obturator, thereby rotating a distal tip of a catheter.

In some embodiments, a fluid is permitted to flow through the first and third fluid pathways. In some embodiments, a fluid is permitted to flow through the first, third and fourth fluid pathways.

In some embodiments, the obturator may one or more slits longitudinally formed through the sidewall of the tubular shaft and in proximity to the distal tip of the obturator, which may be closed. In some embodiments, the slits may have a closed configuration in which a fluid is blocked or prevented from flowing through the slits. In some embodiments, the slits may have an open configuration in which a fluid is permitted to flow through the slits. In some embodiments, the slits may be in the closed configuration when the slits are positioned within the lumen of the catheter. In some embodiments, the slits may be in the open configuration when the slits are positioned distal to the distal tip of the catheter.

In some embodiments, the open configuration of the slits may provide a fifth fluid pathway through the slits. In some embodiments, one or more strips or bands of obturator material may be interposed between the slits and outwardly biased. In some embodiments, the strips or bands of obturator material interposed between the slits may include a resilient material, such as a memory material. In some embodiments, when the slits are positioned distal to the distal tip of the catheter, the slits may open thereby collapsing the position of the distal tip in the proximal direction.

In some embodiments, the obturator may be positioned within the lumen of the catheter such that a fluid is blocked or prevented from flowing through the second, fourth, and fifth fluid pathways. In some embodiments, the obturator may be positioned within the lumen of the catheter such that a fluid is permitted to flow through the fifth fluid pathway. In some embodiments, the obturator may be positioned within the lumen of the catheter such that a fluid is permitted to flow through the second, fourth, and fifth fluid pathways.

It is to be understood that both the foregoing general description and the following detailed description are examples and explanatory and are not restrictive of the present disclosure. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a cross-sectional view of an example fenestrated catheter and an example obturator, illustrating the obturator in a first or distal position, according to some embodiments;

FIG. 2 is a cross-sectional view of the fenestrated catheter and the obturator, illustrating the obturator in a second or proximal position, according to some embodiments;

FIG. 3 is a cross-sectional view of the fenestrated catheter and the obturator, illustrating an example hole within the obturator and the obturator in a first position, according to some embodiments;

FIG. 4 is a cross-sectional view of the fenestrated catheter and the obturator, illustrating the obturator in a second position, according to some embodiments;

FIG. 5 is a cross-sectional view of the fenestrated catheter and another example obturator, illustrating an example closed configuration, according to some embodiments;

FIG. 6 is a cross-sectional view of the fenestrated catheter and the obturator of FIG. 5 positioned to permit fluid flow through an example fenestration of the catheter, according to some embodiments;

FIG. 7 is a cross-sectional view of the fenestrated catheter and the obturator of FIG. 5 in another position to permit fluid flow through an example distal tip and the fenestration of the catheter, according to some embodiments;

FIG. 8 is a cross-sectional view of another example obturator, illustrating an example body and an example shaped guidewire, according to some embodiments;

FIG. 9 is a cross-sectional view of an example vascular access device in the vasculature of a patient, illustrating the obturator of FIG. 8 in a proximal position, according to some embodiments;

FIG. 10 is a cross-sectional view of the vascular access device in the vasculature of a patient, illustrating the obturator of FIG. 8 in a distal position, according to some embodiments;

FIG. 11 is a cross-sectional view of another example obturator, illustrating an example tubular shaft body and an example guidewire, according to some embodiments;

FIG. 12 is a cross-sectional view of an example catheter and the obturator of FIG. 11, according to some embodiments;

FIG. 13 is a cross-sectional view of another example obturator, illustrating an example tubular shaft body with holes in a sidewall of the tubular shaft and an example guidewire, according to some embodiments;

FIG. 14 is a cross-sectional view of an example catheter and the obturator of FIG. 13 in a proximal position, according to some embodiments;

FIG. 15 is a cross-sectional view of an example catheter and the obturator of FIG. 13 in a distal position, according to some embodiments;

FIG. 16 is a cross-sectional view of the fenestrated catheter and another example obturator, illustrating multiple slits in an example closed configuration in which fluid is prevented from flowing through an example hole of the obturator, according to some embodiments;

FIG. 17 is a cross-sectional view of the fenestrated catheter and the obturator of FIG. 16, illustrating the multiple slits in the closed configuration in which fluid is permitted to flow through the hole of the obturator, according to some embodiments; and

FIG. 18 is a cross-sectional view of the fenestrated catheter and the obturator of FIG. 16, illustrating the multiple slits in an example open configuration, according to some embodiments.

### DETAILED DESCRIPTION

As used in the present disclosure, the term “distal” refers to a direction away from a clinician who would place the device into contact with a patient, and nearer to the patient. As used in the present disclosure, the term “proximal” refers to a direction nearer to the clinician who would place the device into contact with the patient, and farther away from the patient.

As previously mentioned, catheter functionality may be impeded for several reasons, particularly when a dwelling time of a catheter within a vasculature is prolonged. For example, the catheter may become occluded at a distal tip and its fenestrations due to the presence of fibrin sheath, thrombus, vein walls, or valves. Applying traction to move or reposition the distal tip within the vein may significantly improve blood draw success and catheter functionality by avoiding such occlusions and obstacles. Further, occlusion may be prevented by selectively opening and closing the tip of the catheter and/or one or more fenestrations. Embodiments described herein disclose a vascular access device that enables aspirations, blood draws, and/or infusions by passively repositioning the tip of the catheter and opening a fluid path and/or by selectively opening and closing at least one of the catheter tip and/or one or more fenestrations.

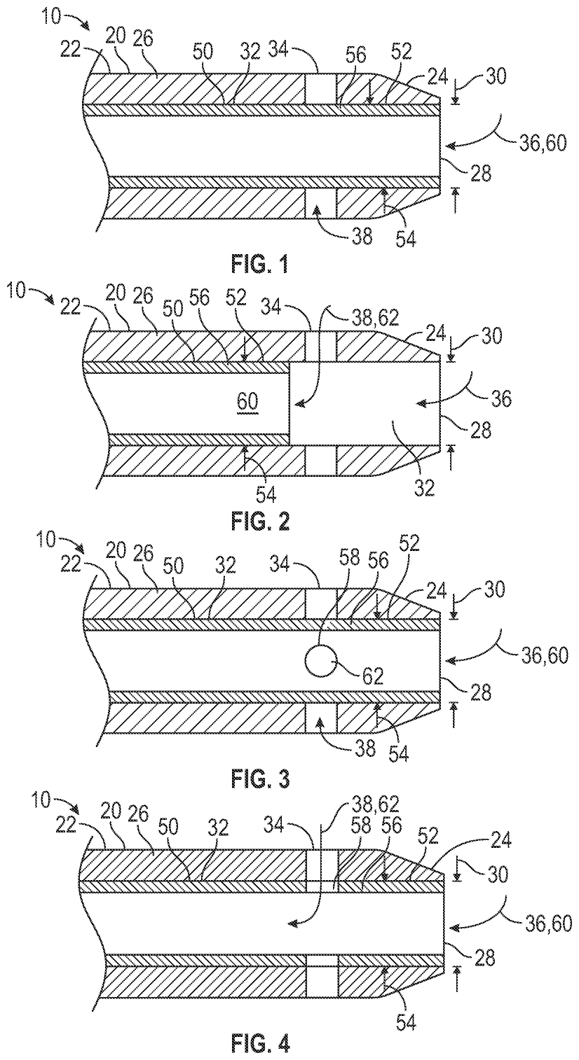

Referring now to FIGS. 1 and 2, in some embodiments, a vascular access device 10 may be configured to prevent occlusion. In some embodiments, the vascular access device 10 may include a catheter 20 and an obturator 50 slidably positioned within the lumen 32 of the catheter 20. In some embodiments, the catheter 20 may include a proximal end 22, a distal tip 24, and a sidewall 26 defining a lumen 32 between the proximal end 22 and the distal tip 24. In some embodiments, the distal tip 24 may include an opening 28 having an inner diameter 30. In some embodiments, the catheter 20 may also include a first fluid pathway 36 through the opening 28. In some embodiments, the sidewall 26 of the catheter 20 has fenestrations 34 which form a second fluid pathway 38 through the fenestrations 34.

In some embodiments, the obturator 50 is slidably positioned within the lumen 32 of the catheter 20 and may include a distal end 52 with an outer diameter 54 configured to form an interference fit with the inner diameter 30 of the distal tip 24 of the catheter 20. In some embodiments, the obturator 50 may include a tubular shaft 56 and a third fluid pathway 60 extending through a length of the tubular shaft 56.

As illustrated in FIG. 1, in some embodiments, the obturator 50 may be positioned within the lumen 32 of the catheter 20 such that a fluid is blocked or prevented from flowing through the second fluid pathway 38. In some embodiments, the obturator 50 may be positioned within the lumen 32 of the catheter 20 such that a fluid is permitted to flow through the first fluid pathway 36 and the third fluid pathway 60.

As illustrated in FIG. 2, in some embodiments, the obturator 50 may be positioned within the lumen 32 of the catheter 20 such that a fluid is permitted to flow through the second fluid pathway 38. In some embodiments, the obturator 50 may be positioned within the lumen 32 of the catheter 20 such that a fluid is permitted to flow through the first fluid pathway 36 and third fluid pathway 60.

Referring now to FIGS. 3 and 4, in some embodiments, the obturator 50 further may include holes 58 in a sidewall of the tubular shaft 56, and a fourth fluid pathway 62 through the holes 58. As illustrated in FIG. 3, in some embodiments, the obturator 50 may be positioned within the lumen of the catheter such that the holes 58 are misaligned with the fenestrations 34 thereby blocking or preventing a fluid from flowing through the second 38 and fourth 62 fluid pathways. In some embodiments, the obturator 50 may be positioned within the lumen 32 of the catheter 20 such that a fluid is permitted to flow through the first fluid pathway 36 and the third fluid pathway 60, as illustrated in FIG. 4.

Referring now to FIGS. 5, 6, and 7, in some embodiments, the distal end 52 of the obturator 50 is solid or closed. With reference to FIG. 5, in some embodiments the obturator 50 may be positioned within the lumen 32 of the catheter 20 such that the opening 28 and the fenestrations 34 of the catheter 20 are blocked thereby preventing a fluid from flowing through the first fluid pathway 36 and second 38 fluid pathways.

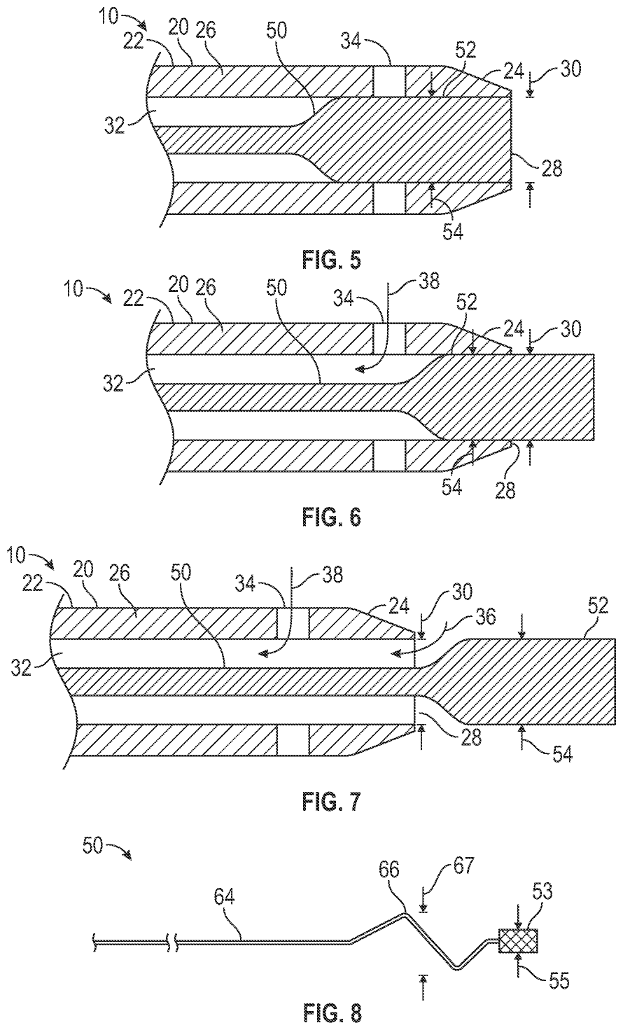

As illustrated in FIG. 6, in some embodiments, the obturator 50 may be positioned within the lumen 32 of the catheter 20 such that the opening 28 (i.e., the first fluid pathway 36) of the catheter is blocked, but the fenestrations 34 are open, thereby permitting a fluid to flow through the second fluid pathway 38.

As illustrated in FIG. 7, in some embodiments, the obturator 50 may be positioned within the lumen 32 of the catheter 20 such that the opening 28 and the fenestrations 34 are open, thereby permitting a fluid to flow through the first fluid pathway 36 and second 38 fluid pathways. In some embodiments, the obturator is advanced distally and proximally within the lumen 32 of the catheter 20 to selectively permit fluid flow through the first fluid pathway 36 and second 38 fluid pathways.

Referring now to FIGS. 8, 9, and 10, in some embodiments, the obturator 50 may include a body 53 with an outer diameter 55 configured to slide within the lumen 32 of the catheter 20. In some embodiments, the outer diameter 55 of the body 53 is configured to form an interference fit with the inner diameter 30 of the catheter 20. In some embodiments, the outer diameter 55 of the body 53 is configured to form an interference fit the inner diameter 30 at the distal tip 24 of the catheter 20. In some embodiments, the obturator 50 may include a guidewire 64 attached to the distal end of the body 53. In some embodiments, the body 53 is a coiled portion of the guidewire 64. In some embodiments, the guidewire 64 further may include a shaped portion 66.

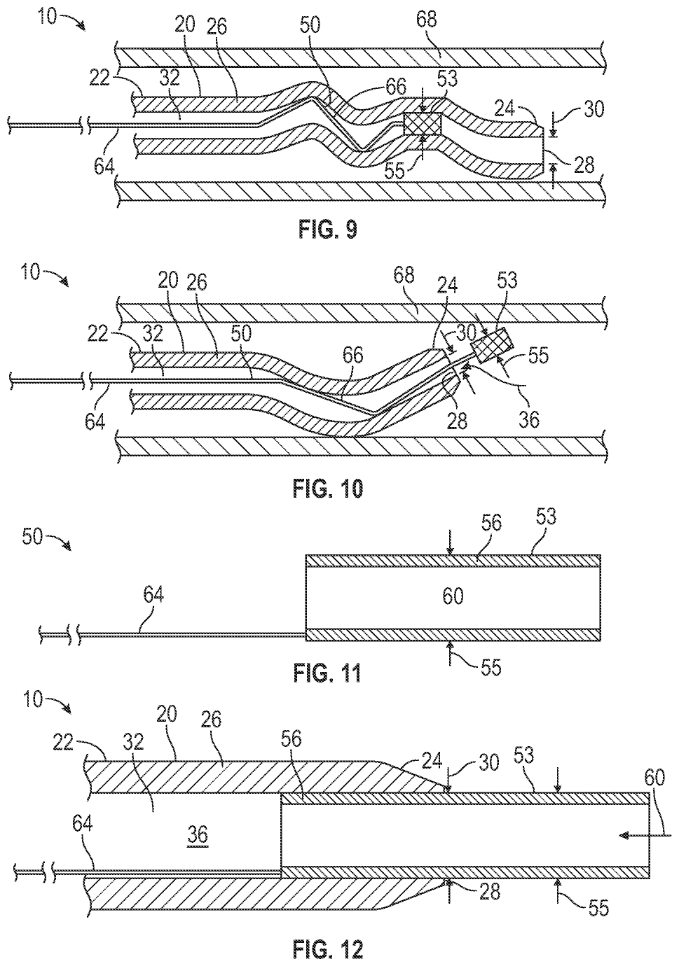

As illustrated in FIGS. 9 and 10, in some embodiments, the shaped portion 66 of the guidewire 64 has a maximum height 67 that is greater than the inner diameter 30 of the catheter 20. In some embodiments, the shaped portion 66 of the guidewire 64 is configured to contact an inner wall surface of the lumen 32 of the catheter 20 to temporarily deform a static conformation of the catheter 20 at a point of contact between the shaped portion 66 of the guidewire 64 and the inner wall surface of the lumen 32 of the catheter 20. In some embodiments, a point of contact between the shaped portion 66 and the inner wall surface of the lumen contacts the inner surface of the lumen 32 at two or more points.

In some embodiments, the shaped portion 66 of the guidewire 64 is S-shaped. In some embodiments, the shaped portion 66 of the guidewire 64 is V-shaped. In some embodiments, the shaped portion 66 of the guidewire 64 is Z-shaped, M-shaped, N-shaped or an equivalent shape that that contacts the inner surface of the catheter lumen at two or more points. In some embodiments, the shaped portion 66 of the guidewire 64 is coiled. In some embodiments, moving or repositioning the guidewire 64 distally, proximally, and/or rotationally causes the distal tip 24 of the catheter 20 to move. In some instances, the moving or repositioning the obturator 50 causes the distal tip 24 of the catheter 20 to move away from an inner sidewall of a patient's vein 68. In some instances, moving or repositioning the obturator 50 causes the distal tip 24 of the catheter 20 to move away from an object or surface occluding the distal tip 24 of the catheter 20.

As illustrated in FIG. 9, in some embodiments, the obturator 50 may be positioned within the lumen 32 of the catheter 20 such that a fluid is blocked or prevented from flowing through the first fluid pathway 36. As the body 53 of the obturator is advanced distally through the lumen 32 of catheter 20, the shaped portion 66 of the guidewire 64 contacts the inner surface of lumen 32 and temporarily deforms the catheter 20. As body 53 advanced distally towards and through distal tip 24, shaped portion 66 may lift and reposition the distal tip 24, as illustrated in FIG. 10. For example, shaped portion 66 may lift and reposition the distal tip 24 away from an interior wall surface of a patient's vein 68, or other surface capable of occluding one or more fluid pathways of catheter 20. In some embodiments, body 53 is advanced distally through opening 28 of distal tip 24 to clear an obstruction from first fluid pathway 36.

Referring now to FIGS. 11 and 12, in some embodiments, the body 53 of the obturator 50 may include a tubular shaft 56 and a third fluid pathway 60 extending through a length of the tubular shaft 56. In some embodiments, moving or repositioning the guidewire 64 of the obturator 50 moves or repositions the body 53 of the obturator 50 relative to the distal tip 24 of the catheter 20. In some embodiments, advancing the guidewire 64 of the obturator 50 distally within the catheter 20 advances the body 53 beyond the distal tip 24 of catheter, thereby effectually extending the length of the catheter 20 within the patient's vein, without requiring that an externally-located portion of the catheter 20 be advanced into the patient. Thus, in some embodiments, an obturator 50 may lengthen an inserted portion of a catheter 20 without further advancing the catheter 20 into the patient.

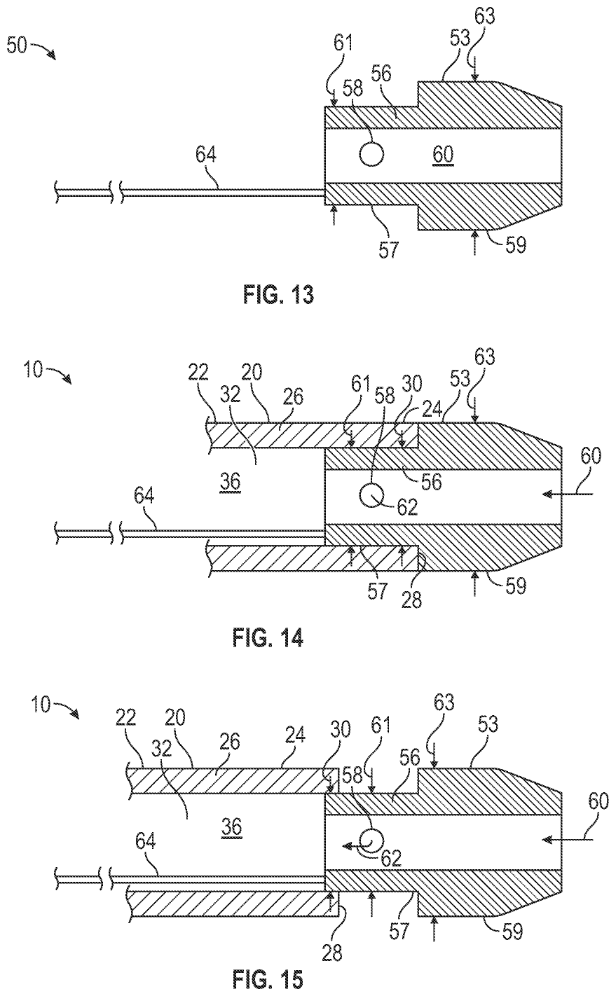

Referring now to FIGS. 13, 14, and 15, in some embodiments, the body 53 of the obturator 50 may include a tubular shaft 56, a third fluid pathway 60 extending through a length of the tubular shaft 56, holes 58 in a sidewall of the tubular shaft 56, and a fourth fluid pathway 62 through the holes 58. In some embodiments, the body 53 of the obturator 50 further comprises a proximal portion 57 with an outer diameter 61 configured to form an interference fit with the inner diameter 30 of the distal tip 24 of the catheter 20 and a distal portion 59 with an outer diameter 63 that is larger than the outer diameter 61 of the proximal portion 57. In some embodiments, the outer diameter 63 of the distal portion 59 of the body 53 is equal, or approximately equal to the outer diameter of the catheter 20.

As illustrated in FIG. 14, in some embodiments, the obturator 50 may be positioned within the lumen 32 of the catheter 20 such that the holes 58 are closed, thereby preventing a fluid from flowing through the fourth fluid pathway 62. As illustrated in FIG. 15, in some embodiments, the obturator 50 may be positioned within the lumen 32 of the catheter 20 such that the holes 58 are open, thereby allowing a fluid to flow through the fourth fluid pathway 62. In some embodiments, the obturator 50 is moved distally or proximally within the lumen 32 of the catheter 20 to open and close the holes 58. In some embodiments, moving the guidewire 64 distally or proximally moves the obturator 50 distally or proximally, thereby opening and closing the holes 58. For example, obturator 50 may be slid distally to position holes 58 distally beyond the distal tip of the catheter 20, thereby unobstructing the fourth fluid pathway 62. In some embodiments, twisting the guidewire 64 rotates the obturator 50 within the lumen 32. For example, obturator 50 may be rotated within lumen 32 to align or misalign holes 58 with one or more fenestrations 34 of catheter 20, thereby obstructing or unobstructing a fluid pathway comprising the fourth fluid pathway 62 and the second fluid pathway 38. In some embodiments, the obturator 50 is positioned within the lumen 32 of a catheter 20 such that a fluid is allowed to flow through the first 36 and third 60 fluid pathways.

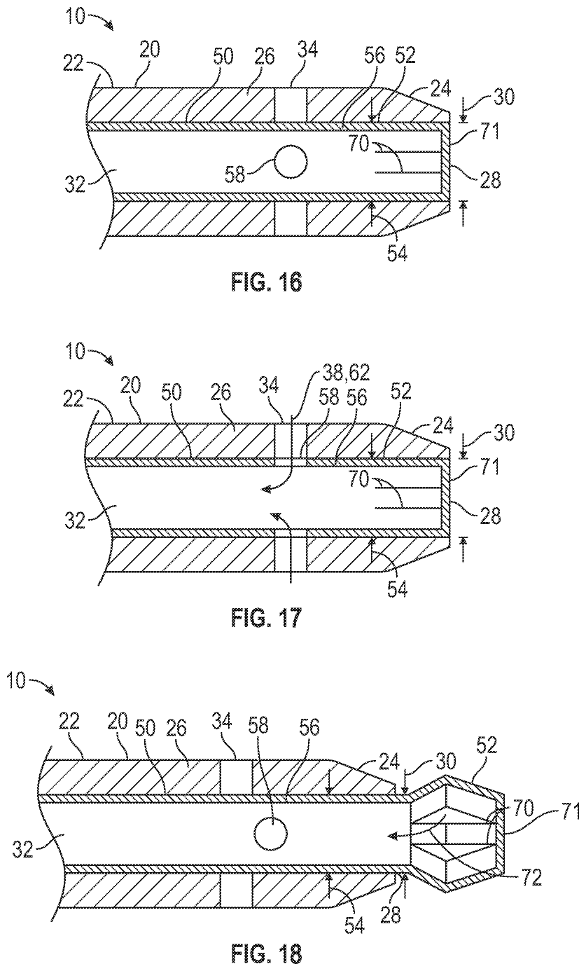

Referring now to FIGS. 16, 17, and 18, in some embodiments, the obturator 50 may include a distal end 52 with an outer diameter 54 configured to form an interference fit with the inner diameter 30 of the distal tip 24 of a catheter 20, a tubular shaft 56, and a solid distal tip 71. In some embodiments, obturator 50 further may include one or more slits 70 longitudinally formed through a sidewall of the tubular shaft 56 proximal the solid distal tip 71. In some embodiments, obturator further may include one or more holes 58 formed through a sidewall of the tubular shaft 56 of the obturator 50, thereby forming a fourth fluid pathway 62 through the holes 58.

In some embodiments, the slits 70 have a closed configuration wherein a fluid is blocked or prevented from flowing through the slits 70 and an open configuration wherein a fluid is permitted to flow through the slits 70. In some embodiments, the slits 70 is in the closed configuration when the slits 70 may be positioned within the lumen 32 of the catheter 20, as illustrated in FIGS. 16 and 17. In some embodiments, the slits 70 is in the open configuration when the slits 70 may be positioned distal to the distal tip 24 of the catheter 20, as illustrated in FIG. 18.

In some embodiments, an open configuration of the slits 70 provides a fifth fluid pathway 72 through the slits 70. In some embodiments, the strips or bands of obturator material interposed between the slits 70 is outwardly biased, such that when the slits 70 are advanced distally beyond distal tip 24, the interposed material expands outwardly, thereby opening slits 70. In some embodiments, the strips or bands of obturator material interposed between the slits 70 may include a resilient material, such as a memory material. In some embodiments, when the slits 70 may be positioned distal to the distal tip 24 of the catheter 20, the slits open thereby collapsing the position of the solid distal tip 71 in the proximal direction.

As illustrated in FIG. 16, in some embodiments, the obturator 50 may be positioned within the lumen 32 of the catheter 20 such that the holes 58 are misaligned with the fenestrations 34 thereby blocking or preventing a fluid from flowing through the second 38 and fourth 62 fluid pathways. In some embodiments, the obturator 50 may be positioned within the lumen 32 of the catheter 20 such that a fluid is blocked or prevented from flowing through the fifth fluid pathway 72.

As illustrated in FIG. 17, in some embodiments, the obturator 50 may be positioned within the lumen 32 of the catheter 20 such that the holes 58 are aligned with the fenestrations 34, thereby permitting a fluid to flow through the second 38 and fourth 62 fluid pathways, and slits 70 are in a closed configuration, thereby preventing fluid flow through the fifth fluid pathway 72. In some embodiments, obturator 50 is axially rotated within lumen 32 to align holes 58 with fenestrations 34. In some embodiments, obturator 50 is advanced distally and/or proximally to align holes 58 with fenestrations 34.

As illustrated in FIG. 18, the slits 70 assume an open configuration when advanced distally beyond the distal tip 24 of catheter 20, thereby permitting a fluid to flow through the fifth fluid pathway 72. In some embodiments, obturator 50 is advanced distally within the lumen 32 to advance the slits 70 beyond the distal tip 24. In some embodiments, a rate of fluid flow through the fifth fluid pathway 72 may be adjusted by selectively positioning the slits 70 within the opening 28. For example, in some embodiments the slits 70 are partially positioned within the opening 28 such that a distal portion of the slits 70 are positioned distally beyond the opening 28 and a proximal portion of the slits 70 are positioned within the lumen 32, such that the slits are only partially opened, thus permitting a limited fluid flow through the fifth fluid pathway 72.

1. A vascular access device, comprising:
a catheter comprising a proximal end, a distal tip comprising an opening having an inner diameter, a sidewall defining a lumen between the proximal end and distal tip, and a first fluid pathway through the opening, the sidewall comprising a fenestration forming a second fluid pathway through the fenestration; and
an obturator slidably positioned within the lumen of the catheter and comprising a distal end with an outer diameter configured to form an interference fit with the inner diameter of the distal tip.

2. The vascular access device of claim 1, further comprising a first position of the obturator within the lumen wherein the first and second fluid pathways are closed.

3. The vascular access device of claim 2, further comprising a second position of the obturator within the lumen wherein the first fluid pathway is closed, and the second fluid pathway is open.

4. The vascular access device of claim 3, further comprising a third position of the obturator within the lumen wherein the first and second fluid pathways are open.

5. The vascular access device of claim 4, wherein the obturator is slid distally within the lumen to move the obturator from the first position to the second position, and from the second position to the third position.

6. The vascular access device of claim 1, wherein the obturator further comprises a tubular shaft and a third fluid pathway extending through a length of the tubular shaft.

7. The vascular access device of claim 6, further comprising a first position of the obturator within the lumen wherein the second fluid pathway is open and comprising a second position of the obturator within the lumen wherein the second fluid pathway is closed.

8. The vascular access device of claim 7, wherein the obturator is slid distally and proximally within the lumen to move the obturator between the first and second positions.

9. The vascular access device of claim 7, wherein the obturator is rotated within the lumen to move the obturator between the first and second positions.

10. The vascular access device of claim 8, further comprising a hole in a sidewall of the tubular shaft, the hole forming a fourth fluid pathway through the hole, wherein when the obturator is in the second position the hole and the fenestration are misaligned, and wherein when the obturator is in the first position the hole and the fenestration are aligned to provide fluid communication through the second and fourth fluid pathways.

11. A vascular access device, comprising:
a catheter comprising a proximal end, a distal tip comprising an opening having an inner diameter, a sidewall defining a lumen with an inner wall surface between the proximal end and distal tip, and a first fluid pathway through the opening; and
an obturator slidably positioned within the lumen of the catheter and comprising a body with an outer diameter configured to form an interference fit with the inner diameter of the distal tip and a guidewire attached to a distal end of the body.

12. The vascular access device of claim 11, wherein the guidewire further comprises a shaped portion, wherein the shaped portion has a maximum height greater than the inner diameter and is configured to temporarily deform a static conformation of the catheter at a point of contact between the shaped portion and an inner wall surface of the lumen.

13. The vascular access device of claim 12, further comprising a first position of the obturator within the lumen wherein the first fluid pathway is closed and comprising a second position of the obturator within the lumen wherein the first fluid pathway is open.

14. The vascular access device of claim 11, wherein the body further comprises a tubular shaft having a second fluid pathway through a length of the tubular shaft.

15. A vascular access device, comprising:
a catheter comprising a proximal end, a distal tip comprising an opening having an inner diameter, a sidewall defining a lumen between the proximal end and distal tip, and a first fluid pathway through the opening, the sidewall comprising a fenestration forming a second fluid pathway through the fenestration; and
an obturator slidably positioned within the lumen of the catheter and comprising a distal end with an outer diameter configured to form an interference fit with the inner diameter of the distal tip, a tubular shaft, a solid distal tip, a slits longitudinally formed through a sidewall of the tubular shaft proximal the solid distal tip, a third fluid pathway through the slits, a hole in a sidewall of the tubular shaft, and a fourth fluid pathway through the hole.

16. The vascular access device of claim 15, further comprising a first position of the obturator within the lumen wherein the third and fourth fluid pathways are closed, comprising a second position of the obturator within the lumen wherein the third fluid pathway is closed, and the fourth fluid pathway is open, comprising a third position of the obturator within the lumen wherein the third fluid pathway is open, and the fourth fluid pathway is closed, and comprising a fourth position of the obturator within the lumen wherein the third and fourth fluid pathways are open.

17. The vascular access device of claim 16, wherein the obturator is slid distally, proximally, and/or rotated within the lumen to move the obturator into the first, second, third, and fourth positions.

18. The vascular access device of claim 15, further comprising a resilient material interposed between the slits.
